(19) United States
(12) Patent Application Publication
(10) Pub. No.: US 2010/0139653 A1
(43) Pub. Date: Jun. 10, 2010

(54) Ventilator interface

(76) Inventor: Judy Schloss, Lester Prairie, MN (US)

Correspondence Address: Judy Schloss, 6243 180th Street, Lester Prairie, MN, 55354, US

(21) Appl. No.: 12/587,044

(22) Filed: Oct. 1, 2009

Related U.S. Application Data
(60) Provisional application No. 61/101,774, filed on Oct. 1, 2008.

(51) Int. Cl. A61M 16/10

(52) U.S. Cl. 128/203.12

(57) ABSTRACT

An adaptor for providing fluid communication between a counter equipped measured dose inhaler (MDI) canister and a ventilator circuit includes a housing, a receiver tube for engaging the valve stem of the canister, and a tab for engaging the counter mechanism. The housing defines a primary lumen which extends between an input limb and an output limb. The limbs are configured to mate with the tubing of a ventilator circuit. The receiver tube defines a receiver lumen therethrough and extends from the exterior of the housing and into the primary lumen. The receiver lumen provides fluid communication between the exterior of the housing and the primary lumen.

### CROSS-REFERENCE TO RELATED APPLICATIONS

None.

### BACKGROUND OF THE INVENTION

1. Field of the Invention

Embodiments of the invention are directed to ventilator systems and more particularly to ventilator systems and/or their components adapted to interface with a metered dose inhaler (MDI).

2. Description of the Related Art

Ventilators are automatic mechanical devices designed to provide all or part of the work the body must produce to move gas into and out of the lungs. Though many types of ventilators are known in the art, features common to nearly all conventional types include a number of working components that cooperate with one another to ensure the desired ventilation of the patient's lungs is realized. More specifically, a conventional ventilator includes a stable attachment (also called an interface or accessory) of the device to the patient; a source of energy to drive the device; a control system to make it perform appropriately; and a means of monitoring the performance of the device and the condition of the patient.

Conventional ventilators deliver gas to the patient through a set of flexible conduits or tubes called a patient circuit. Typically, the ventilator includes two tubes: one associated with exhalation and the other associated with inhalation (there are some ventilator systems which include only a single tube). The circuit connects the ventilator to either an endotracheal or tracheostomy tube that extends into the patient's throat (in the case of an invasive ventilation), or a mask covering the mouth and nose or just the nose (in the case of a noninvasive ventilation).

Often when a patient is connected to a ventilator, it is necessary to deliver therapeutic agents (drugs, medicine, etc) into the lungs. A common way to do this is to include in the circuit pathway a mechanism that will receive the valve stem of a canister of medicine, such as those canisters typically associated with a metered dose inhaler (MDI). Such a receiver mechanism provides a convenient and user friendly way to directly inject medicine into the lungs of the patient by utilizing the inhalation event of the ventilator.

MDIs are well known devices, and are often referred to simply as inhalers. They are devices that help deliver a predetermined amount (dose) of medication to the pulmonary system, and particularly the lungs. They are commonly used to treat asthma, chronic obstructive pulmonary disease (COPD), and/or other respiratory problems.

A hand held inhaler or MDI often comprises two major components: a canister and a mouth piece body. The canister itself includes a metering dose valve with an actuating stem, and contains the aerosol propellant and medication or medications. The mouth piece body contains a valve stem receptor that defines a port to engage the stem of the canister, and the mouth piece itself, to which the user places to their mouth to receive the medication contained in the canister. The mouth piece commonly includes a dust cap to prevent contamination.

Once assembled the patient or care provider uses the inhaler by pressing down on the top of the canister while supporting lower portion of the body or mouthpiece, thus releasing a mist of medication that is then inhaled into the lungs. When utilized in a ventilator circuit, such as described above, the valve stem of the canister is typically mated directly to a receiver (without the mouth piece body) on the inhalation tube of the circuit. Direct compression of the canister against the receiver expels medicine into the circuit and then into the lungs of the patient.

A recent trend in the make-up of MDIs is to include a counter device that is incorporated directly into assembly of the canister and/or valve stem. The counter is provided in order to enable the user to see the number of doses expelled from, or remaining in, the canister. An example of an MDI equipped with a counter mechanism is illustrated in PRIOR ART FIGS. 1a.-1e.

As shown, the counter equipped MDI includes the canister assembly 10 and the mouth piece body 20. The mouth piece body 20 defines a canister receiving region 21 and a valve stem neck 22, which receives the valve stem 11. The canister 10 includes a counter mechanism 12 which at least partially surrounds the valve stem 11. The counter 10 includes a display mechanism 13 such as a gear and/or spindle driven wheel or wheels that display the number of doses remaining in the canister. The display mechanism 13 is configured to be repeatedly engaged and actuated by a tab, protrusion, or other device 23 within the mouth piece 20. Each time the engagement mechanism 12 is engaged by protrusion 23, the display 13 progressively displays each dose expelled from the canister 10 when the MDI is used in the manner described above.

Unfortunately, the valve stems of canister assemblies that include counters will not effectively mate with the receiver mechanisms or other known adaptors of ventilator circuits (an example of which is illustrated in PRIOR ART FIG. 2). Moreover, attempts to modify counter equipped MDI canisters, so that they may be utilized with current receivers, will often result in significant damage to either or both structures, rendering the canister (and the medicine contained therein) un-useable.

It is recognized that counter equipped MDI canisters are becoming more common place, and will most likely replace non-counter equipped canisters. As a result, care givers and individual vented patients are forced to adopt the counter equipped MDI canisters despite having no way of conveniently injecting the medicine of the canister into the ventilator circuit. Thus, a need exists to provide an adaptor mechanism for conveniently mating a counter equipped MDI canister, or the MDI itself, directly to the circuit of a ventilator.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

### BRIEF SUMMARY OF THE INVENTION

In light of the above, at least some embodiments of the present invention are directed to a mechanical interface which allows a counter equipped MDI canister to interface into a ventilator circuit without the need to modify or remove the counter from the canister.

In at least one embodiment of the invention an interface mechanism is provided that will adaptively mate to the valve stem of a counter equipped MDI.

These and other embodiments which characterize the invention are pointed out with particularity in the claims annexed hereto and forming a part hereof. However, for further understanding of the invention, its advantages and objectives obtained by its use, reference should be made to the drawings which form a further part hereof and the accompanying descriptive matter, in which there are illustrated and described further embodiments of the invention.

### BRIEF DESCRIPTION OF THE SEVERAL VIEWS OF THE DRAWING(S)

A detailed description of the invention is hereafter described with specific reference being made to the drawings.

FIG. 1a is a side view of a PRIOR ART MDI device.

FIG. 1b is a diagrammatic view of the PRIOR ART canister assembly shown in PRIOR ART FIG. 1a.

FIG. 1c is a top down internal view of the PRIOR ART mouth piece shown in PRIOR ART FIG. 1a.

FIG. 1d is a side view of the canister assembly shown in PRIOR ART FIGS. 1a and 1b.

FIG. 1e is an alternate side view of the canister assembly shown in PRIOR ART FIGS. 1a and 1b.

FIG. 2 is a partial perspective view of a PRIOR ART ventilator circuit.

FIG. 3 is a solid perspective view of an embodiment of the invention.

FIG. 4 is a solid cross-sectional perspective view of the embodiment shown in FIG. 3.

FIG. 5 is a perspective component (see-through) view of the embodiment shown in FIG. 3.

FIG. 6 is a cross-sectional component perspective view of the embodiment shown in FIG. 3

FIG. 7 is a top down view of the embodiment shown in FIG. 3.

FIG. 8 is a front component view of the embodiment shown in FIG. 3.

FIG. 9 is a side component view of the embodiment shown in FIG. 3.

FIG. 10 is a rear component view of the embodiment shown in FIG. 3.

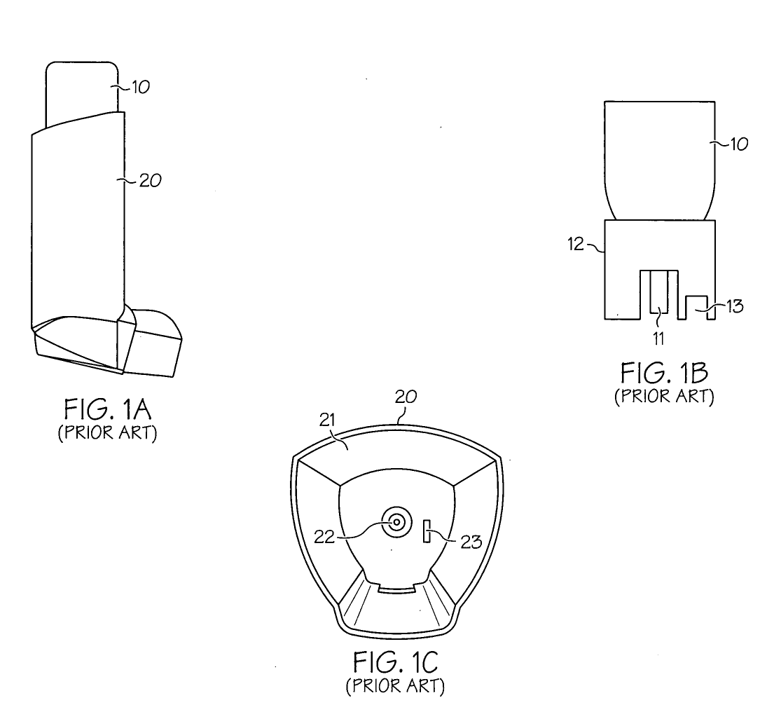

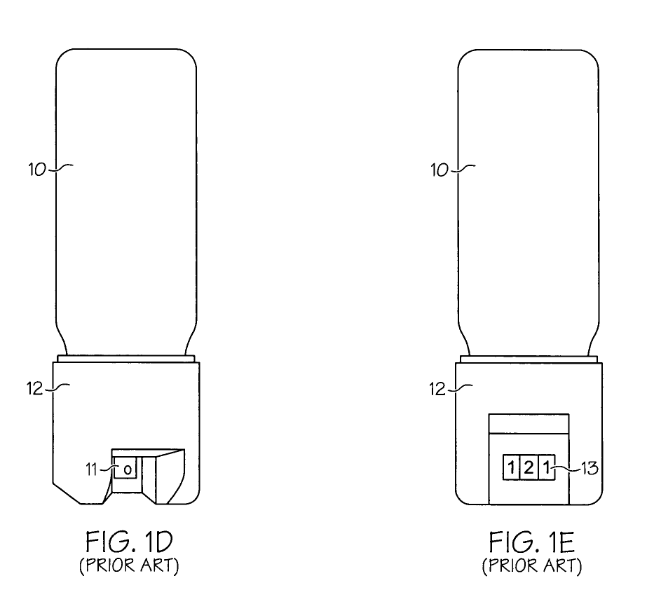

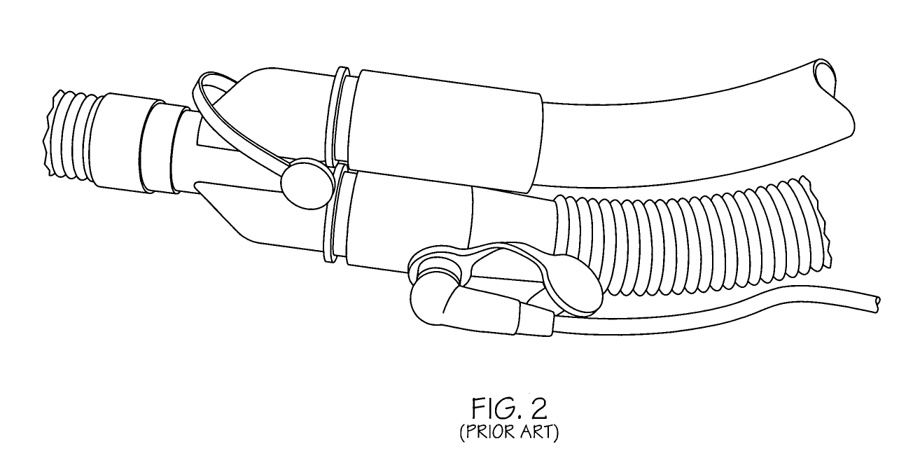

### DETAILED DESCRIPTION OF THE INVENTION

While this invention may be embodied in many different forms, there are described in detail herein specific embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

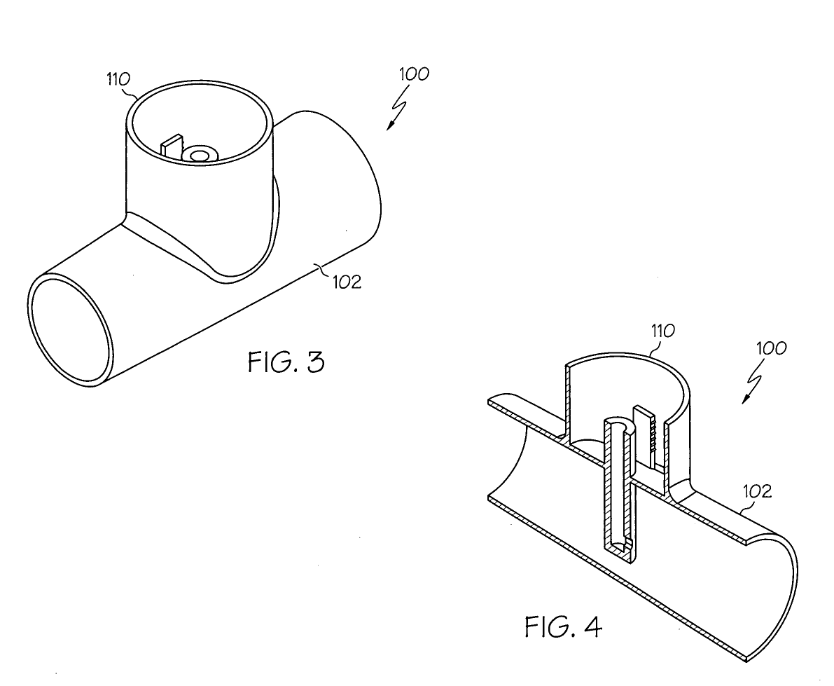

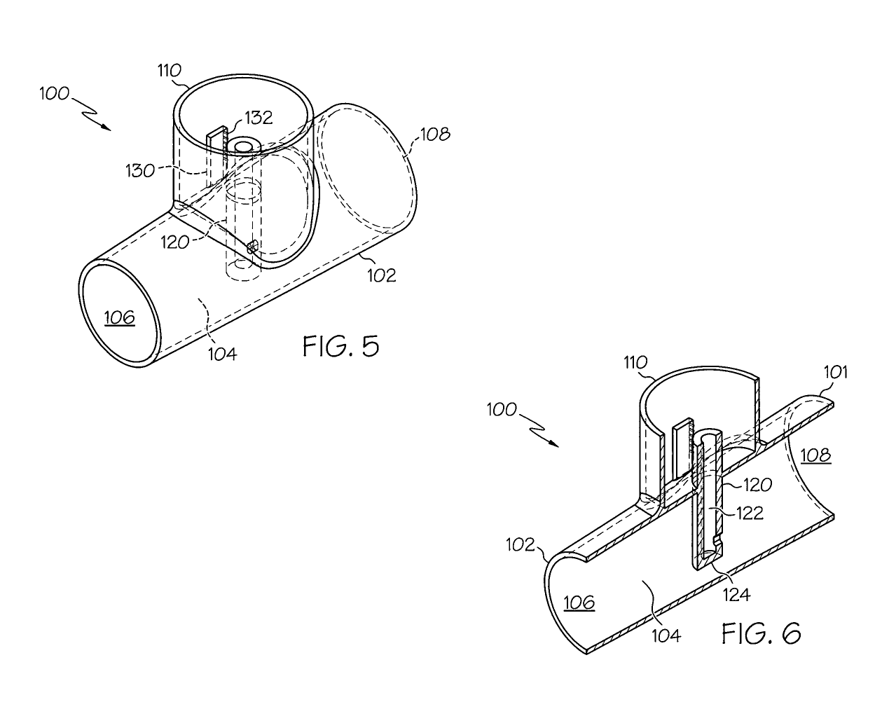

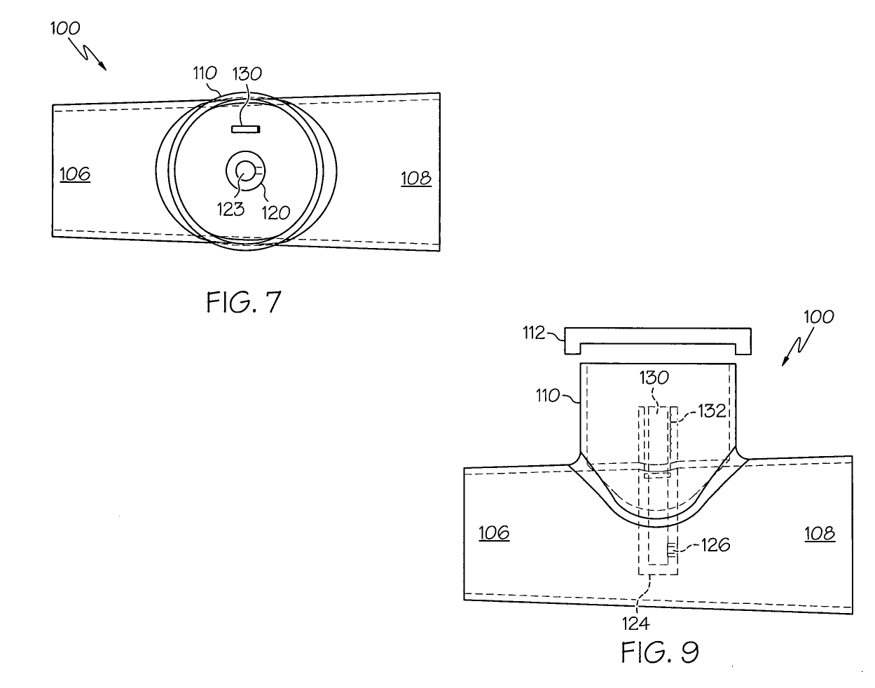

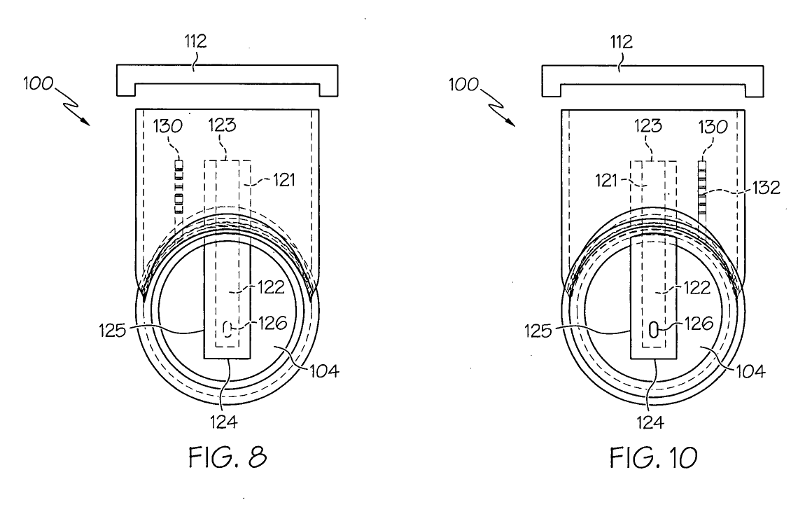

Turning to the embodiment of the present invention, illustrated in FIGS. 3-10 a unique line ventilator circuit adaptor, or t-connector 100 is shown. Adaptor 100 is configured to be inserted directly into a ventilator circuit and allow a direct interface with of a counter equipped MDI canister 10 therein.

Circuit adaptor 100 is a substantially T-shaped housing 102 of material such as plastic, metal, etc., which defines a primary lumen 104 through which the fluid (e.g. breathable air, oxygen and/or oxygen mixtures, etc.) of the ventilator circuit can freely flow. The housing of the primary lumen 104 is divided into an input limb 106 and an output limb 108. The external diameter of each limb may be the same, however in at least one embodiment, at least a portion of the exterior of the input limb 106 has a narrower diameter than the exterior of the output limb 108. In the embodiment shown herein the input limb 106 is narrower to allow the ventilator tubing to be engaged over or on the outside surface of the housing, whereas the output limb 108 engages the exterior of the ventilator circuit tubing.

On the exterior 101 of the primary lumen housing 102 is positioned an MDI receiver collar 110. The collar has an interior diameter sufficient to removeably receive a counter equipped MDI canister 10, such as of the type previously shown and described above. The collar is further configured to removeably receive a cap or cover 112 which overlays the collar opening 114 when the canister 10 is not in use or present.

In some embodiments the cap and collar interface includes threads to allow the cap to be screwed into place. In some embodiments the cap and collar interface includes one or more flanges, indentations and/or protrusions to allow the cap and collar to frictionally engage one another with a “snap-fit”. Regardless of the particular mechanism used to engaged the cap 112 to the collar 110, the interface between the two desirably forms an air tight seal.

Within the collar 110 is positioned an external portion 121 of valve stem receiver 120. In at least one embodiment, at least the exterior portion 121 of the receiver 120 is centrally positioned within the collar 110, though in some embodiments the receiver can be offset if desired.

The receiver 120 is an elongate tube of the same or different material as the housing 102 and defines a receiver lumen 122. An exterior portion 121 of the receiver 120 includes an opening 123 in communication with the receiver lumen 122. The opening 123 has an inner diameter sized to receive and engaged the valve stem 11 of a counter equipped MDI canister 10 of the type previously shown and described. In some embodiments the exterior portion 121 of the receiver 120 is sized and configured such that the external surface of the receiver lumen 122 engages the internal surface of the canister valve stem 11.

The receiver tube 120 has a length that extends from within the collar 110, through the housing 102, and into the primary lumen 104. The portion of the receiver 120 within the primary lumen is referred to as the interior portion 125 of the receiver 120. The receiver 120 terminates at an interior end 124 within the primary lumen 104. Adjacent to the interior end 124, the receiver 120 defines an interior opening or port 126. Port 126 may be defined by any portion of the receiver 120, but in at least one embodiment port 126 is defined by the region of the tube generally facing the output limb 108 of the primary lumen 104. By facing in this direction, drug or other therapeutic agent delivered from canister 10 (through opening 123 and lumen 122) will enter the primary lumen 104 and be directed into the ventilator circuit in the same direction of the inhalant flowing to the patient.

In some embodiments the receiver tube 120 includes at least one unidirectional valve. Such a valve is positioned within or adjacent to the receiver lumen 122 to allow a therapeutic agent to enter the primary lumen 104 (via the receiver lumen 122) but prevents the therapeutic agent or other fluid (breathable air, etc.) present in the ventilator circuit from escaping back through the receiver lumen 122.

In order for the present circuit adapter 100 to be fully effective with counter equipped MDI canisters, the adaptor must include an engagement tab 130 configured to engage and actuate the counter mechanism 12 of the canister 10. Such a tab 130 is positioned on the external surface 101 of the primary lumen housing 102, adjacent to receiver tube 120 and with the perimeter of the collar 110.

The particular orientation, spacing from the receiver tube, size, shape, length, and other physical characteristics of the tab 130 are selected depending on the type of canister 10 to be utilized. For example, in at least one embodiment the tab 130 includes along one lateral face 132 a plurality of engagement teeth 134. These teeth are numbered, sized and arranged to engage the gear/spindle (not shown) of the counter mechanism 12 of the type of canister 10 shown in the PRIOR ART figures.

In at least one embodiment the tab 130 is identical in size and shape to the engagement device 23 of the mouth piece 20 depicted in the PRIOR ART figures.

In use adaptor 100 is inserted into the inspiratory limb of a ventilator circuit with the output limb 108 proximal to the patient. Once in position, a counter equipped MDI canister 10 is inserted within the collar 110. The canister valve stem 11 engages the valve stem receiver 120 of the adaptor 100 and the canister's counter mechanism 12 engages the tab 130. When positioned in this manner the canister 10 can be compressed toward the adaptor 100 causing the valve stem 11 to release a single dose of medication from the canister, while the tab 130 simultaneously engages and actuates the particular gear/spindle or other system of the counter mechanism 12. This actuation will result in the display mechanism 13 advancing to visually display that a medication dose has been expelled from the canister.

This completes the description of the invention. Those skilled in the art may recognize other equivalents to the specific embodiment described herein which equivalents are intended to be encompassed by the claims attached hereto.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

What is claimed is as follows:

1. An adaptor for providing fluid communication between a counter equipped measured dose inhaler (MDI) canister and a ventilator circuit, the adaptor comprising:
a housing, the housing defining a primary lumen, the primary lumen extending between in input limb and an out put limb;
a receiver tube, the receiver tube extending externally through the housing and into the primary lumen, the receiver tube defining a receiver lumen, the receiver lumen having an external opening and an internal opening in fluid communication with the primary lumen, an external portion of the receiver tube constructed and arranged to removeably engage and actuate a valve stem of a counter equipped MDI canister; and
an engagement tab, the engagement tab positioned external to the housing and adjacent to the receiver tube, the engagement tab constructed and arranged to engage and actuate a counter mechanism of the counter equipped MDI canister.

2. The adaptor of claim 1 further comprising a collar, the collar defining a perimeter, the collar externally engaged to the housing.

3. The adaptor of claim 2 wherein the external portion of the receiver tube is positioned within the perimeter of the collar.

4. The adaptor of claim 3 wherein the external portion of the receiver tube is centrally positioned within the perimeter of the collar.

5. The adaptor of claim 1 further comprising a cap, the cap constructed and arranged to be removeably and sealingly engaged to the collar.
